(19) United States
(12) Patent Application Publication
(10) Pub. No.: US 2023/0128861 A1
(43) Pub. Date: Apr. 27, 2023

(54) Improved Methods and Compositions for Processing Manure

(71) Applicant: Locus IP Company, LLC, Solon, OH (US)

(72) Inventor: Sean FARMER, Ft. Lauderdale, FL (US)

(21) Appl. No.: 17/770,162

(22) PCT Filed: Jul. 15, 2021
(86) PCT No.: PCT/US2021/041781

§ 371 Date: Apr. 19, 2022

Related U.S. Application Data
(60) Provisional application No. 63/052,074, filed on Jul. 15, 2020.

(51) Int. Cl. C05F 17/20; A01C 3/02; C05F 3/00; C05G 3/90; C05G 3/50

(52) U.S. Cl. CPC C05F 17/20; A01C 3/026; C05F 3/00; C05G 3/90; C05G 3/50

(57) ABSTRACT

The subject invention provides improved methods for processing livestock waste, namely, for solid-liquid separation of manure, utilizing microbe-based products. In preferred embodiments, microorganisms and/or microbial surfactants are utilized to improve solid-liquid separation of livestock manure in ways that enhance the value of manure-based fertilizers to farmers and reduces greenhouse gas and other polluting emissions resulting from manure storage.

### CROSS-REFERENCE TO RELATED APPLICATION

This application claims priority to U.S. Provisional Patent Application No. 63/052,074, filed Jul. 15, 2020, which is incorporated herein by reference in its entirety.

### BACKGROUND OF THE INVENTION

With livestock production comes a variety of environmental and health considerations, including management and processing of manure. The average dairy cow can produce more than 100 pounds of manure per day. Mishandling of manure can pose a risk to nearby ground and surface water through runoff and leaching, as well as the possibility of leaks or spills into nearby rivers and lakes. Additionally, greenhouse gas emissions and odors from manure can pollute the atmosphere. For example, methanogenic and sulfate-reducing microbes in manure can convert organic materials into methane and hydrogen sulfide, respectively, while nitrogen in feces and urine, and uric acid in poultry manure, can lead to formation of ammonia and nitrous oxide when the manure begins to decompose. Accordingly, proper handling techniques must be employed to ensure environmental and health standards are met.

Manure and urine produced in barns and confined animal feeding operations (CAFOs) is typically collected in tanks under the floors where the animals stand, or is removed either by shoveling, flushing, scraping or via vacuuming systems. When flushed with water, this “slurry” manure can be transported using pumps, which sometimes contain chopping or grinding mechanisms to reduce the solid particle size and prevent plugging. The slurry manure is collected in storage facilities, such as a lagoon, pond or storage tank and/or is transported to an agricultural crop for direct use.

Processing of manure can involve multiple steps, depending on what end purpose the manure will serve. For example, manure can be treated to kill pathogens, removed of sand and fiber particles for reuse as animal bedding, and/or anaerobically digested. Anaerobic digestion by microorganisms helps break down simple sugars, volatile fatty acids and alcohols into carbon dioxide and methane, which can reduce solid particle size and improve transport and separation, in addition to producing a source of biogas to power trucks and buildings. This can be performed before and/or after an important process in manure management: solid-liquid separation, or dewatering.

Essentially, solid-liquid separation of manure is the physical process of separating slurry manures into two fractions liquid and solid. Soluble components such as plant-available nitrogen, phosphates, sodium, chloride, ammonium and potassium, tend to concentrate in the liquid phase. Metals and organically-bound and/or insoluble components, such as organic nitrogen, organic phosphorous, and calcium phosphate, tend associate with larger solid particles, such as bedding material and undigested fibers. The solids fraction, sometimes called “sludge,” is often subjected to further dewatering to reduce the moisture content even more.

Solid-liquid separation is widely used in the livestock industry as a means of, for example, reducing organic loading in a lagoon or waste storage pond; reducing sludge buildup in a lagoon; removing solids from slurry manure to facilitate pumping of manure liquids; generating separated solids for use as an ingredient to make compost or to recycle animal bedding; producing treatable wastewater to flush manure from animal housing areas; improving the uniformity of solids and plant nutrients in separated liquids; removing excess nutrients, such as phosphorus and nitrogen, from separated liquids; and improving the balance of nutrients in the separated liquids to better match crop requirements.

Separation techniques can be categorized into methods that exploit particle density differences, which include gravity settling, centrifuges, and hydrocyclones, and methods that exploit particle size differences, which include stationary inclined screens, in-channel flighted conveyor screens, rotating screens, screw presses, belt presses, and rotary presses.

Some of these mechanical methods of separation can be enhanced by introducing a coagulation step. The suspended colloidal solids in manure carry a negative surface charge, which disperses the particles and keeps them in suspension. A coagulant, typically a cationic substance such as metal salts of aluminum, iron and/or calcium, causes coagulation of these suspended particles, and may concurrently cause precipitation of dissolved nutrients, such as phosphate and nitrogen. By precipitating phosphates and nitrogen, they can be collected with the coarser solids, thereby enhancing the nutrient content of the solids fraction and reducing their presence in the liquid fraction. This provides farms with natural fertilizer as well as clean process water for, e.g., irrigation. Coagulants can also reduce the pH of manure, which can be useful for reducing ammonia emissions.

It is sometimes helpful in the separation process to add a flocculation step as well. With flocculation, a substance with the ability to combine particles into a larger, denser floc is added to the manure. These flocs of particles are then more easily removed by mechanical means. A high molecular weight polymer, such as a polyacrylamide (PAM) or chitosan, is typically used for flocculation.

While coagulation and flocculation are helpful tools for increasing the efficiency of manure separation and dewatering, the result is often the presence of residual salts and/or polymers in the separated manure fractions. This decreases the value of these fractions for use as, for example, fertilizers and soil amendments, because these compounds can impair the health of soil and crops when applied to a field. Additionally, some coagulants, such as those containing iron, may increase production of the greenhouse gas methane by bacteria that are present in manure. Thus, coagulation and/or flocculation can increase the operating costs and reduce the positive environmental impacts of manure management to a livestock producer.

Manure management is both practically and environmentally beneficial. It can help reduce the risk of contaminating surface water, groundwater or drinking water; reduce greenhouse gas emissions; improve soil quality, structure and water-holding capacity; and recycle crucial nutrient compounds. From an economic standpoint, manure management may also reduce the need for producers to buy fertilizer to spread on grazing fields and crops.

Current technologies for solids and nutrient separation, however, have inherent limitations, are costly to operate, and result in the use of large quantities of fuel and labor in order to provide solid and liquid effluents that can, for example, be recycled, are environmentally acceptable to spread on farmlands, and/or can be used as potable water. Thus, improved methods for separation and dewatering of manure are needed.

### BRIEF SUMMARY OF THE INVENTION

The subject invention provides improved methods for manure management. More specifically, the subject invention provides improved methods for solid-liquid separation of manure using microbe-based products. Advantageously, the methods of the subject invention are environmentally-friendly, operational-friendly and cost effective.

In preferred embodiments, the subject invention provides methods for solid-liquid separation of manure, wherein a microbe-based product comprising a microbial biosurfactant and/or a beneficial microorganism is applied to the manure, thereby promoting the formation of a liquid fraction and a solids fraction.

In certain embodiments, the liquid fraction comprises water and soluble compounds, including, for example, some plant-available nitrogen, phosphates, sodium, chloride, ammonium, and/or potassium.

In certain embodiments, the solids fraction comprises organic material, undigested plant matter, bedding fibers, microbial cells, and other insoluble materials, such as, for example, organic nitrogen, organic phosphorous, and calcium phosphate.

In certain embodiments, the solids fraction is collected from the treated manure using mechanical separation methods known in the art, such as, for example, centrifuging, screening, and the like. Advantageously, the subject methods can be used to increase the total solids (TS) content and/or reduce the moisture content of the solids fraction (percent by mass), compared to what is achieved using mechanical separation without prior treatment according to the subject methods. In certain embodiments, the subject methods can reduce the moisture content of manure to below 50%, preferably below 40%, more preferably below 25% by mass using safe and environmentally-friendly techniques and ingredients.

In some embodiments, the solids fraction and/or the liquid fraction can be re-treated with the microbe-based products according to the subject methods in order to achieve further separation of solids, including dissolved solids, and liquids.

In certain embodiments, the subject methods can be used to thicken (i.e., dewater) slurry manure to be treated in an anaerobic digester. By reducing the water content, a higher volume of manure can be placed into an anaerobic digester at one time, thereby increasing the throughput efficiency of treatment.

The manure treated according to the subject methods can be raw manure, liquid manure, slurry manure, and/or a separated fraction of manure (e.g., a liquid or solids fraction). In some embodiments, the manure, or fraction thereof, has previously been subjected to processing such as, for example, blending or chopping, anaerobic digestion, decontamination, mechanical separation, gravity separation or separation according to the subject methods.

In preferred embodiments, the biosurfactant utilized according to the subject methods is a glycolipid. In some embodiments, a combination of different biosurfactants is utilized. The biosurfactant(s) can be in a purified form, or in a crude form comprising residual materials from the culture in which the biosurfactant was produced.

In certain preferred embodiments, the biosurfactant is a sophorolipid. Sophorolipids (SLP) are glycolipids that comprise a sophorose consisting of two glucose molecules, linked to a fatty acid by a glycosidic ether bond. SLP can be acetylated on the 6 and/or 6′ positions of the sophorose residue. One terminal or subterminal hydroxylated fatty acid is β-glycosidically linked to the sophorose molecule. The fatty acid of a SLP can have one or more unsaturated bonds. SLP can exist in either monomeric or dimeric forms. They also can be either lactonic, where the carboxyl group in the fatty acid side chain and the sophorose moiety form a cyclic ester bond; or the acidic form, or linear form, where the ester bond is hydrolyzed.

Other biosurfactants can also be used, including, for example, other glycolipids (e.g., rhamnolipids, mannosylerythritol lipids, cellobiose lipids and trehalose lipids), lipopeptides (e.g., surfactin, iturin, fengycin, athrofactin and lichenysin), fatty acid esters, flavolipids, phospholipids (e.g., cardiolipins), and high molecular weight polymers such as lipoproteins, lipopolysaccharide-protein complexes, and polysaccharide-protein-fatty acid complexes.

In certain embodiments, the methods utilize a beneficial microorganism in combination with and/or in place of the biosurfactant. The microbe can be in the form of vegetative cells, spores and/or a combination thereof.

Preferably, the beneficial microorganism is capable of producing biosurfactants and/or other metabolites useful for separating manure and/or for controlling detrimental microorganisms in manure, e.g., methanogens and/or sulfate-reducing bacteria (SRB). Exemplary beneficial microbes include Bacillus amyloliquefaciens, Bacillus subtilis, Starmerella bombicola, Wickerhamomyces anomalus, Meyerozyma guilliermondii, Saccharomyces chlororaphis, Saccharomyces cerevisiae, Debaryomyces hansenii, and others.

In a specific exemplary embodiment, the methods utilize a sophorolipid in combination with a strain of Bacillus, such as, for example, B. amyloliquefaciens strain NRRL B-67928 or B. subtilis strain NRRL B-68031 (“B4”). The amount of sophorolipid must not exceed an amount that inhibits survival of the microorganism.

In some embodiments the beneficial microorganism(s) produce other growth by-products, including, e.g., enzymes, biopolymers, solvents, acids, proteins, polyketides, amino acids, terpenes, fatty acids, and/or other metabolites useful for enhancing animal, soil, plant and/or environmental health. More specifically, the growth by-products may be useful for, e.g., digesting and/or composting manure solids, killing detrimental microbes and pathogens in manure, promoting soil and plant health in manure-based fertilizers and soil amendments, and/or reducing greenhouse gas and other polluting emissions from manure.

Advantageously, the subject methods are useful for producing liquid manure fractions that can be used directly as, e.g., field irrigation water, animal drinking water, and water for washing animal housing and agricultural equipment. In certain embodiments, the liquid fraction comprises some of the microbial biosurfactant and/or microorganism, thereby providing the added benefits thereof for enhancing animal, soil, plant and/or environmental health.

In some embodiments, the liquid fraction can be transported for traditional municipal and/or agricultural wastewater treatment and recycling.

Advantageously, the subject methods are also useful for producing solids manure fractions that can be used directly for, e.g., composting, animal bedding, combustible biofuels, fertilizers and soil amendments. In certain embodiments, the solids fraction comprises some of the microbial biosurfactant and/or microorganism, thereby providing the added benefits thereof for enhancing animal, soil, plant and/or environmental health.

In certain embodiments, the subject methods can be used for reducing and/or replacing traditional coagulation and/or flocculation materials, which utilize metal salts and/or polymers that can contaminate and reduce the value of manure-based products, such as fertilizers.

Advantageously, the subject methods can be used as part of a sustainable agriculture and livestock system, which uses environmentally-friendly, biodegradable materials to reduce manure volume and recycle valuable materials present in manure, all while reducing greenhouse gas emissions from manure.

### DETAILED DESCRIPTION

The subject invention provides improved methods for manure management. More specifically, the subject invention provides improved methods for solid-liquid separation of manure using microbe-based products. Advantageously, the methods of the subject invention are environmentally-friendly, operational-friendly and cost effective.

In preferred embodiments, the subject invention provides methods for solid-liquid separation of manure, wherein a microbe-based product comprising a microbial biosurfactant and/or a beneficial microorganism is applied to the manure, thereby promoting the formation of a liquid fraction and a solids fraction.

### Selected Definitions

The subject invention utilizes “microbe-based compositions,” meaning compositions that comprise components that were produced as the result of the growth of microorganisms or other cell cultures. Thus, the microbe-based composition may comprise the microbes themselves and/or by-products of microbial growth. The microbes may be in a vegetative state, in spore form, in any other form of microbial propagule, or a mixture of these. The microbes may be planktonic or in a biofilm form, or a mixture of both. The by-products of growth may be, for example, metabolites (e.g., enzymes and/or biosurfactants), cell membrane components, proteins, and/or other cellular components. The microbes may be intact or lysed. The cells may be absent, or present at, for example, a concentration of at least 1×10³, 1×10⁴, 1×10⁵, 1×10⁶, 1×10⁷, 1×10⁸, 1×10⁹, 1×10¹⁰, 1×10¹¹, 1×10¹², or 1×10¹³ or more CFU per milliliter of the composition.

The subject invention further provides “microbe-based products,” which are products that are to be applied in practice to achieve a desired result. The microbe-based product can be simply a microbe-based composition. Alternatively, the microbe-based product may comprise further ingredients that have been added. These additional ingredients can include, for example, stabilizers, buffers, carriers (e.g., water or salt solutions), added nutrients to support further microbial growth, non-nutrient growth enhancers and/or agents that facilitate tracking of the microbes and/or the composition in the environment to which it is applied. The microbe-based product may also comprise mixtures of microbe-based compositions. The microbe-based product may also comprise one or more components of a microbe-based composition, e.g., a biosurfactant, which have been processed in some way such as, but not limited to, filtering, centrifugation, lysing, drying, purification and the like.

As used herein, a “biofilm” is a complex aggregate of microorganisms, such as bacteria, wherein the cells adhere to each other and/or to a surface via an extracellular polysaccharide matrix. The cells in biofilms are physiologically distinct from planktonic cells of the same organism, which are single cells that can float or swim in liquid medium.

As used herein, an “isolated” or “purified” nucleic acid molecule, polynucleotide, polypeptide, protein, organic compound such as a small molecule (e.g., those described below), or other compound is substantially free of other compounds, such as cellular material, with which it is associated in nature. For example, a purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. A purified or isolated microbial strain is removed from the environment in which it exists in nature. Thus, the isolated strain may exist as, for example, a biologically pure culture, or as spores (or other forms of the strain) in association with a carrier.

In certain embodiments, purified compounds are at least 60% by weight the compound of interest. Preferably, the preparation is at least 75%, more preferably at least 85%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism (e.g., a growth by-product) or a substance necessary for taking part in a particular metabolic process. A metabolite can be an organic compound that is a starting material, an intermediate in, or an end product of metabolism. Examples of metabolites can include, but are not limited to, enzymes, acids, solvents, alcohols, polyketides, proteins, carbohydrates, vitamins, minerals, microelements, amino acids, biopolymers, and biosurfactants.

As used herein, “modulate” means to cause an alteration (e.g., increase or decrease). Such alterations are detected by standard art known methods.

As used herein, the term “plurality” refers to any number or amount greater than one.

As used herein, “reduction” refers to a negative alteration, and the term “increase” refers to a positive alteration, wherein the negative or positive alteration is at least 0.25%, 0.5%, 1%, 5%, 10%, 15%, 20%, 25%, 30%, 35%, 40%, 45%, 50%, 55%, 60%, 70%, 75%, 80%, 85%, 90%, 95%, or 100%.

As used herein, “reference” refers to a standard or control condition.

As used herein, “surfactant” refers to a compound that lowers the surface tension (or interfacial tension) between phases. Surfactants act as, e.g., detergents, wetting agents, emulsifiers, foaming agents, and dispersants. A “biosurfactant” or “biological amphiphilic molecule” is a surface active molecule produced by a living organism and/or using naturally-derived substances.

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19, or 20 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims. All references cited herein are hereby incorporated by reference.

### Methods of Processing Manure

The subject invention provides improved methods for manure management. More specifically, the subject invention provides improved methods for solid-liquid separation of manure using microbe-based products. Advantageously, the methods of the subject invention are environmentally-friendly, operational-friendly and cost effective alternatives to current methods for manure management. Furthermore, in certain embodiments, the remediation of livestock waste according to the subject methods can have beneficial effects for the animals themselves, such as, for example, increased litter sizes and reduced stress and/or mortality due to overall improvements in living conditions.

In preferred embodiments, the subject invention provides methods for solid-liquid separation of manure, wherein a microbe-based product comprising a microbial biosurfactant and/or a beneficial microorganism is applied to the manure, thereby promoting the formation of a liquid fraction and a solids fraction.

As used herein, “applying” means contacting a composition with manure such that the composition can have a desired effect on the manure, e.g., solid-liquid separation. For example, the microbe-based products according to the subject invention can be poured or injected into manure, a manure lagoon, waste pond, tailing pond, tank or other storage facility where livestock manure is stored and/or treated. In some embodiments, the mixture is mixed for an amount of time to provide even distribution of the microbe-based product throughout the manure, for example, from 1 minute to 6 hours, or 10 minutes to 1 hours, depending on the volume of manure being treated.

In some embodiments, the mixture is allowed to sit undisturbed for an amount of time after mixing, for example, from 1 minute to 72 hours, such that gravity can initiate the separation of solids and liquids in the manure.

In certain embodiments, the solids fraction is collected from the treated manure using mechanical separation methods known in the art, such as, for example, centrifuge, and hydrocyclones, stationary inclined screens, in-channel flighted conveyor screens, rotating screens, screw presses, belt presses, and rotary presses.

In certain embodiments, the liquid fraction comprises water and soluble compounds, including, for example, some plant-available nitrogen, phosphates, sodium, chloride, ammonium, and/or potassium.

In certain embodiments, the solids fraction comprises organic material, undigested plant matter, bedding fibers, microbial cells, and other insoluble materials, such as, for example, organic nitrogen, organic phosphorous, and calcium phosphate.

Advantageously, in some embodiments, the subject methods can be used to increase the total solids (TS) content and/or reduce the moisture content of the solids fraction (percent by mass), compared to what is achieved using mechanical separation without prior treatment according to the subject methods.

For example, in some embodiments, the TS content of the solids fraction is 0.01% to 99%, 0.1% to 95%, 1.0% to 90%, 5.0% to 80%, 10% to 70%, 15% to 60%, 20% to 50%, 25% to 40%, or 30% to 35% greater than solids fractions obtained using mechanical separation without prior treatment with the microbe-based products of the subject invention.

In some embodiments, the moisture content of the solids fraction is 0.01% to 99%, 0.1% to 95%, 1.0% to 90%, 5.0% to 80%, 10% to 70%, 15% to 60%, 20% to 50%, 25% to 40%, or 30% to 35% less than solids fractions obtained using mechanical separation without prior treatment with the microbe-based products of the subject invention.

Advantageously, in some embodiments, the subject methods can be used to increase the rate of solid-liquid separation, meaning, decrease the time it takes to achieve a desired moisture content reduction for manure, compared to what is achieved using mechanical separation without prior treatment according to the subject methods.

For example, in some embodiments, the amount of time to achieve a manure moisture content of 50% or less can be reduced by at least 1%, 5%, 10%, 15%, 20%, 30%, 40%, 50%, 60%, 70%, 80%, 90%, 95% or at least 99% compared to the amount of time required using mechanical separation without prior treatment according to the subject methods.

In some embodiments, the solids fraction and/or the liquid fraction can be re-treated with the microbe-based products according to the subject methods in order to achieve further separation of solids, including dissolved solids, and liquids.

In certain embodiments, the subject methods can be used to thicken (i.e., dewater) slurry manure to be treated in an anaerobic digester. By reducing the water content, a higher volume of manure comprising volatile solids for microbial digestion can be placed into an anaerobic digester at one time, thereby increasing the throughput efficiency of treatment.

The manure treated according to the subject methods can be raw manure, solid manure, liquid manure, slurry manure, and/or a separated fraction of manure (e.g., a liquid or solids fraction). In some embodiments, the manure, or fraction thereof, has previously been subjected to processing such as, for example, blending or chopping, anaerobic digestion, decontamination, mechanical separation, gravity separation or separation according to the subject methods.

In preferred embodiments, the subject methods comprise applying a microbe-based product comprising a biosurfactant to manure. In one embodiment, the biosurfactant has been purified from the cultivation medium in which it was produced. Alternatively, in one embodiment, the growth by-product is utilized in crude form. The crude form can comprise, for example, a liquid supernatant resulting from cultivation of a microbe that produces the growth by-product of interest, which may include residual live or inactive cells and/or nutrients.

Biosurfactants are a structurally diverse group of surface-active substances produced by microorganisms. Biosurfactants are biodegradable and can be produced using selected organisms on renewable substrates. Most biosurfactant-producing organisms produce biosurfactants in response to the presence of a hydrocarbon source (e.g., oils, sugar, glycerol, etc.) in the growing media.

All biosurfactants are amphiphiles consisting of two parts; a polar (hydrophilic) moiety and non-polar (hydrophobic) group. The hydrocarbon chain of a fatty acid acts as the common lipophilic moiety of a biosurfactant molecule, whereas the hydrophilic part is formed by ester or alcohol groups of neutral lipids, by the carboxylate group of fatty acids or amino acids (or peptides), organic acids in the case of flavolipids, or, in the case of glycolipids, by a carbohydrate.

Due to their amphiphilic structure, biosurfactants increase the surface area of hydrophobic water-insoluble substances, and increase the water bioavailability of such substances. Biosurfactants accumulate at interfaces, thus reducing interfacial tension and leading to the formation of aggregated micellar structures in solution. The ability of biosurfactants to form pores and destabilize biological membranes permits their use as antibacterial, antifungal, and hemolytic agents. Combined with the characteristics of low toxicity and biodegradability, biosurfactants are advantageous for use in a variety of application, including in manure treatment.

Biosurfactants according to the subject methods can be, for example, glycolipids (e.g., sophorolipids, rhamnolipids, mannosylerythritol lipids, cellobiose lipids, and trehalose lipids), lipopeptides (e.g., surfactin, iturin, fengycin, arthrofactin and lichenysin), flavolipids, phospholipids (e.g., cardiolipins), fatty acid esters, and high molecular weight polymers such as lipoproteins, lipopolysaccharide-protein complexes, and polysaccharide-protein-fatty acid complexes.

The one or more biosurfactants can further include any one or a combination of: a modified form, derivative, fraction, isoform, isomer or subtype of a biosurfactant, including forms that are biologically or synthetically modified.

In preferred embodiments, the biosurfactant utilized according to the subject methods is a glycolipid. In some embodiments, a combination of different biosurfactants is utilized. The biosurfactant(s) can be in a purified form, or in a crude form comprising residual materials from the culture in which the biosurfactant was produced.

In certain preferred embodiments, the biosurfactant is a sophorolipid. Sophorolipids (SLP) are glycolipids that comprise a sophorose consisting of two glucose molecules, linked to a fatty acid by a glycosidic ether bond. SLP can be acetylated on the 6 and/or 6′ positions of the sophorose residue. One terminal or subterminal hydroxylated fatty acid is β-glycosidically linked to the sophorose molecule. The fatty acid of a SLP can have one or more unsaturated bonds. SLP can exist in either monomeric or dimeric forms. They also can be either lactonic, where the carboxyl group in the fatty acid side chain and the sophorose moiety form a cyclic ester bond; or the acidic form, or linear form, where the ester bond is hydrolyzed.

In certain embodiments, the methods comprise applying about 0.01 to 10,000 ppm, 0.1 to 5,000 ppm, 0.5 to 1,000 ppm, 1.0 to 750 ppm, 1.5 to 500 ppm, 2.0 to 250 ppm, 2.5 to 150 ppm, or 3.0 to 100 ppm biosurfactant with respect to the amount of manure.

In some embodiments, the biosurfactant helps reduce the interfacial tension between manure solids and liquids, thereby promoting separation thereof. In some embodiments, this is achieved due to the amphiphilic nature of the biosurfactant, which helps sequester and flocculate charged dissolved solids and/or promotes coalescence of water molecules.

In some embodiments, the biosurfactant can directly inhibit a detrimental microorganism in the manure.

In certain embodiments, the methods utilize a beneficial microorganism in combination with and/or in place of the biosurfactant. The microbe can be in the form of vegetative cells, spores and/or a combination thereof. Preferably, the beneficial microorganism is capable of producing biosurfactants and/or other metabolites useful for, e.g., separating manure and/or controlling detrimental microorganisms.

As used herein, a “beneficial” microbe is one that confers a benefit to manure processing, rather than one that is detrimental. Benefits can include, for example, direct digestion of solids, production of metabolites that help degrade solids, direct control of detrimental microorganisms, and/or support of other beneficial microorganisms.

A “detrimental” microorganism is one that causes direct or indirect harm to humans or animals, to the environment, and/or to the manure treatment process, for example, by killing beneficial microorganisms or producing harmful growth by-products, including greenhouse gases and other pollutants, such as methane, carbon dioxide, nitrous oxide, ammonia/ammonium and/or hydrogen sulfide. Detrimental microorganisms can also include pathogenic organisms, which, if not removed from manure, can cause harm to other living organisms or the environment.

Examples of detrimental microorganisms according to the subject methods include methanogens, which are microorganisms that produce methane gas as a by-product of metabolism. Methanogens are archaea that can be found in the digestive systems and metabolic waste of ruminant animals and non-ruminant animals (e.g., pigs, poultry and horses). Examples of methanogens include, but are not limited to, Methanobacterium spp. (e.g., M. formicicum), Methanobrevibacter spp. (e.g., M. ruminantium), Methanococcus spp. (e.g., M. paripaludis), Methanoculleus spp. (e.g., M. bourgensis), Methanoforens spp. (e.g., M. stordalenmirensis), Methanofollis liminatans, Methanogenium wolfei, Methanomicrobium spp. (e.g., M. mobile), Methanopyrus kandleri, Methanoregula boonei, Methanosaeta spp. (e.g., M. concilii, M. thermophile), Methanosarcina spp. (e.g., M. barkeri, M. mazeii), Methanosphaera stadtmanae, Methanospirillium hungatei, Methanothermobacter spp., and/or Methanothrix sochngenii.

While methanogenesis can be useful for biogas production, the production of methane from stored manure or manure applied to crop fields results in undesirable, polluting emissions of methane and other greenhouse gases into the atmosphere.

Additional examples of detrimental microorganisms include sulfate-reducing bacteria and archaea (SRB), e.g., Proteobacteria, Deltaproteobacteria, Desulfobacterales, Desulfovibrionales, Syntrophobacterales, Desulfotomaculum, Desulfosporomusa, Desulfosporosinus, Thermodesulfovibrio, Thermodesulfobacteria, Thermodesulfobium, Archaeoglobus, Thermocladium, Caldivirga, Desulfuromonas, Desulfovibrio, Desulfurella, Geobacter, Pelobacter, Wolinella, Campylobacter, Shewanella, Sulfurospirillum, Geospirillum, Thermococcales, Thermoproteales, Pyrodictales, and Sulfolobales.

SRB obtain energy by oxidizing organic compounds or molecular hydrogen (H₂) while reducing sulfate (SO²⁻⁴) to hydrogen sulfide (H₂S). Many SRB can also reduce other oxidized inorganic sulfur compounds, such as sulfite, thiosulfate, or elemental sulfur to hydrogen sulfide. H₂S can become an air pollutant and can be extremely toxic for humans if inhaled at certain concentrations.

The beneficial microorganisms of the subject invention may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In one specific embodiment, the composition comprises about 1×10³ to about 1×10¹³, about 1×10⁴ to about 1×10¹², about 1×10⁵ to about 1×10¹¹, or about 1×10⁶ to about 1×10¹⁰ CFU/g of each species of beneficial microorganism present in the composition.

In one embodiment, the composition comprises about 1 to 100% beneficial microorganisms and/or microbial cultures total by volume, about 10 to 90%, or about 20 to 75%.

In certain preferred embodiments, the composition comprises one or more bacteria and/or growth by products thereof. The bacteria can be, for example, a Myxococcus sp. (e.g., M. xanthus), and/or one or more Bacillus spp. bacteria. In certain embodiments, the Bacillus spp. are B. amyloliquefaciens, B. subtilis and/or B. licheniformis. Bacteria can be used in spore form, as vegetative cells, and/or as a mixture thereof.

In one embodiment, the composition comprises B. amyloliquefaciens. In a preferred embodiment, the strain of B. amyloliquefaciens is B. amyloliquefaciens NRRL B-67928 (“B. amy”). In another specific embodiment, the composition comprises B. subtilis strain B4 (NRRL B-68031). In a specific exemplary embodiment, the composition comprises both B. amy and B4.

In certain embodiments, B. amy is particularly advantageous due to its ability to produce a mixture of lipopeptide biosurfactants that is unique when compared with biosurfactant production capabilities of reference strains of B. amyloliquefaciens, as well as all Bacillus spp. This lipopeptide mixture comprises surfactin, lichenysin, fengycin and iturin A. In some embodiments, B. amy produces greater total amounts of biosurfactants compared to reference strains of Bacillus amyloliquefaciens.

In some embodiments, B. amy survives and grows under high saline conditions and at temperatures of 55° C. or higher. The strain is also capable of growing under anaerobic conditions. The B. amy strain can also be used for producing enzymes that degrade or metabolize starches.

In some embodiments, B. amy is capable of producing glycolipid biosurfactants, phytase, organic acids, nitrogen fixation enzymes and/or growth hormones.

In some embodiments, B. amy can produce spores that remain viable in an animal's digestive tract and, in some embodiments, after being excreted in the animal's waste.

In certain embodiments, the composition comprises a strain of Bacillus subtilis. In preferred embodiments, the strain is B. subtilis “B4” (NRRL B-68031). Advantageously, in some embodiments, strain B4 can produce lipopeptide biosurfactants in enhanced amounts, particularly surfactin. Advantageously, in some embodiments, B4 and/or the enhanced amounts of surfactin that it produces, can be especially helpful for enhanced disruption of methanogenic biofilms in livestock digestive tracts and waste.

In some embodiments, B4 is “surfactant over-producing.” For example, the strain may produce at least 0.1-10 g/L, e.g., 0.5-1 g/L biosurfactant, or, e.g., at least 10%, 25%, 50%, 100%, 2-fold, 5-fold, 7.5 fold, 10-fold, 12-fold, 15-fold or more compared to other B. subtilis bacteria. For example, in some embodiments, ATCC 39307 can be used as a reference strain.

In a specific exemplary embodiment, the methods utilize a sophorolipid in combination with B. amy and/or with strain B4. The amount of sophorolipid must not exceed an amount that inhibits survival of the microorganism.

Cultures of the B. amy and B4 strains have been deposited with the Agricultural Research Service Northern Regional Research Laboratory (NRRL), 1400 Independence Ave., S.W., Washington, DC, 20250, USA. The B. amy deposit has been assigned accession number NRRL B-67928 by the depository and was deposited on Feb. 26, 2020. The B4 deposit has been assigned accession number NRRL B-68031 by the depository and was deposited on May 6, 2021.

Each of the subject cultures has been deposited under conditions that assure that access to the culture will be available during the pendency of this patent application to one determined by the Commissioner of Patents and Trademarks to be entitled thereto under 37 CFR 1.14 and 35 U.S.0 122. The deposit is available as required by foreign patent laws in countries wherein counterparts of the subject application, or its progeny, are filed. However, it should be understood that the availability of a deposit does not constitute a license to practice the subject invention in derogation of patent rights granted by governmental action.

Further, each of the subject culture deposits will be stored and made available to the public in accord with the provisions of the Budapest Treaty for the Deposit of Microorganisms, i.e., it will be stored with all the care necessary to keep it viable and uncontaminated for a period of at least five years after the most recent request for the furnishing of a sample of the deposit, and in any case, for a period of at least 30 (thirty) years after the date of deposit or for the enforceable life of any patent which may issue disclosing the culture. The depositor acknowledges the duty to replace the deposit should the depository be unable to furnish a sample when requested, due to the condition of the deposit. All restrictions on the availability to the public of the subject culture deposit will be irrevocably removed upon the granting of a patent disclosing it.

In one embodiment, the beneficial microorganisms are yeasts and/or fungi. Yeast and fungus species suitable for use according to the current invention, include Acaulospora, Acremonium chrysogenum, Aspergillus, Aureobasidium (e.g., A. pullulans), Blakeslea, Candida (e.g., C. albicans, C. apicola, C. batistae, C. bombicola, C. floricola, C. kuoi, C. riodocensis, C. nodaensis, C. stellate), Cryptococcus, Debaryomyces (e.g., D. hansenii), Entomophthora, Hanseniaspora (e.g., H. uvarum), Hansenula, Issatchenkia, Kluyveromyces (e.g., K. phaffii), Lentinula spp. (e.g., L. edodes), Meyerozyma (e.g., M. guilliermondii), Monascus purpureus, Mortierella, Mucor (e.g., M. piriformis), Penicillium, Phythium, Phycomyces, Pichia (e.g., P. anomala, P. guilliermondii, P. occidentalis, P. kudriavzevii), Pleurotus (e.g., P. ostreatus P. ostreatus, P. sajorcaju, P. cystidiosus, P. cornucopiae, P. pulmonarius, P. tuberregium, P. citrinopileatus and P. flabellatus), Pseudozyma (e.g., P. aphidis), Rhizopus, Rhodotorula (e.g., R. bogoriensis); Saccharomyces (e.g., S. cerevisiae, S. boulardii, S. torula), Starmerella (e.g., S. bombicola), Torulopsis, Thraustochytrium, Trichoderma (e.g., T. reesei, T. harzianum, T. viridae), Ustilago (e.g., U. maydis), Wickerhamiella (e.g., W. domericqiae), Wickerhamomyces (e.g., W. anomalus), Williopsis (e.g., W. mrakii), Zygosaccharomyces (e.g., Z. bailii), and others.

In certain specific embodiments, the composition comprises one or more fungi and/or one or more growth by-products thereof. The fungi can be, for example, Pleurotus spp. fungi, e.g., P. ostreatus (oyster mushrooms), Lentinula spp. fungi, e.g., L. edodes (shiitake mushrooms), and/or Trichoderma spp. fungi, e.g., T. viridae. The fungi can be in the form of live or inactive cells, mycelia, spores and/or fruiting bodies. The fruiting bodies, if present, can be, for example, chopped and/or blended into granules and/or a powder form.

In certain specific embodiments, the composition comprises one or more yeasts and/or one or more growth by-products thereof. The yeast(s) can be, for example, Wickerhamomyces anomalus (e.g., strain NRRL Y-68030), Saccharomyces spp. (e.g., S. cerevisiae and/or S. boulardii), Debaryomyces hansenii, Starmerella bombicola, Meyerozyma guilliermondii, Pichia occidentalis, Monascus purpureus, and/or Acremonium chrysogenum. The yeast(s) can be in the form of live or inactive cells or spores, as well as in the form of dried and/or doimant cells (e.g., a yeast hydrolysate).

In preferred embodiments, the beneficial microorganism(s) produce a biosurfactant. In some embodiments the beneficial microorganism(s) produce other growth by-products, including, e.g., enzymes, biopolymers, solvents, acids, proteins, polyketides, amino acids, terpenes, fatty acids, and/or other metabolites useful for enhancing animal, plant, soil and/or environmental health. The growth by-products preferably can be useful for, e.g., digesting and/or composting manure solids, killing pathogens in manure, promoting soil and plant health in manure-based fertilizers and soil amendments, and/or reducing greenhouse gas and other polluting emissions from manure (e.g., methane, hydrogen sulfide, carbon dioxide, nitrous oxide and ammonia/ammonium).

In certain embodiments, the method comprises applying a germination enhancer to manure for enhancing germination of spore-form microorganisms that may be used in subject methods. In specific embodiments, the germination enhancers are amino acids, such as, for example, L-alanine and/or L-leucine. In one embodiment, the germination enhancer is manganese.

In one embodiment, the method comprises applying one or more fatty acids to the manure. The fatty acids can be produced by the beneficial microorganism(s), and/or produced separately and included as an additional component. In certain preferred embodiments, the fatty acid is a saturated long-chain fatty acid, having a carbon backbone of 14-20 carbons, such as, for example, myristic acid, palmitic acid or stearic acid. In some embodiments, a combination of two or more saturated long-chain fatty acids is included in the composition. In some embodiments, a saturated long-chain fatty acid can inhibit methanogenesis and/or increase cell membrane permeability of methanogens.

In some embodiments, the methods can comprise applying additional components known to reduce methane production, such as, for example, seaweed (e.g., Asparagopsis taxiformis); kelp; 3-nitrooxypropanol; anthraquinones; ionophores (e.g., monensin and/or lasalocid); polyphenols (e.g., saponins, tannins); Yucca schidigera extract (steroidal saponin-producing plant species); Quillaja saponaria extract (triterpenoid saponin-producing plant species); organosulfurs (e.g., garlic extract); flavonoids (e.g., quercetin, rutin, kaempferol, naringin, and anthocyanidins; bioflavonoids from green citrus fruits, rose hips and black currants); carboxylic acid; and/or terpenes (e.g., d-limonene, pinene and citrus extracts).

Advantageously, the subject methods are useful for producing liquid manure fractions that can be used directly as, e.g., field irrigation water, animal drinking water, and water for washing animal housing and agricultural equipment. In certain embodiments, the liquid fraction comprises some of the microbial biosurfactant and/or the beneficial microorganism, thereby providing the added benefits thereof for animal, soil, plant and/or environmental health.

In some embodiments, the liquid manure fractions can be used for irrigating a crop or field, wherein a manure liquid fraction obtained according to the subject methods is applied to the crop or field, wherein the presence of the biosurfactant or a biosurfactant-producing microbe in the irrigation liquid enhances the movement of the water throughout soil and into plant roots, thereby, in certain embodiments, enhancing water use efficiency for growers.

In some embodiments, the liquid fraction can be transported for traditional municipal wastewater treatment and recycling.

Advantageously, the subject methods are also useful for producing solids manure fractions that can be used directly for, e.g., composting, animal bedding, combustible biofuels, fertilizers and soil amendments. In certain embodiments, the solids fraction comprises some of the microbial biosurfactant and/or beneficial microorganism, thereby providing the added benefits thereof for animal, soil, plant and/or environmental health.

In certain embodiments, the subject methods can be used for reducing and/or replacing traditional coagulation and/or flocculation materials, which utilize metal salts and/or polymers that can contaminate and reduce the value of manure-based products, such as fertilizers.

In certain embodiments, this can also help reduce the amount of carbon dioxide, nitrous oxide, ammonia, hydrogen sulfide and/or methane that are produced from manure storage facilities by reducing the number of microbes that produce those compounds, for example, methanogens and/or SRB. The methods can also facilitate increased decomposition of manure components, thereby reducing manure storage capacity, GHG emissions, water contamination, and odor nuisance that comes with manure storage. Advantageously, this benefits environmental health, animal health, and the health of workers and local citizens.

Furthermore, in some embodiments, applying the microbe-based product(s) to manure enhances the value of the manure as an organic fertilizer due to the ability of the beneficial microorganism(s) to inoculate the soil of a field or crop to which the manure is eventually applied. The microorganisms and their growth by-products can improve soil biodiversity, enhance rhizosphere properties, and enhance plant growth and health, which can lead to, for example, a reduced need for nitrogen-rich synthetic fertilizers (and thus, a reduction in ammonia and nitrous oxide emissions resulting from use of synthetic fertilizers).

Advantageously, the subject methods can be used as part of a sustainable agriculture and livestock system, which uses environmentally-friendly, biodegradable materials to reduce manure volume and recycle valuable materials present in manure, all while reducing greenhouse gas emissions from manure.

Production of Microorganisms and/or Microbial Growth By-Products

The subject invention utilizes methods for cultivation of microorganisms and production of microbial metabolites and/or other by-products of microbial growth. The subject invention further utilizes cultivation processes that are suitable for cultivation of microorganisms and production of microbial metabolites on a desired scale. These cultivation processes include, but are not limited to, submerged cultivation/fermentation, solid state fermentation (SSF), and modifications, hybrids and/or combinations thereof.

As used herein “fermentation” refers to cultivation or growth of cells under controlled conditions. The growth could be aerobic or anaerobic. In preferred embodiments, the microorganisms are grown using SSF and/or modified versions thereof.

In one embodiment, the subject invention provides materials and methods for the production of biomass (e.g., viable cellular material), extracellular metabolites, residual nutrients and/or intracellular components.

The microbe growth vessel used according to the subject invention can be any fermenter or cultivation reactor for industrial use. In one embodiment, the vessel may have functional controls/sensors or may be connected to functional controls/sensors to measure important factors in the cultivation process, such as pH, oxygen, pressure, temperature, humidity, microbial density and/or metabolite concentration.

In a further embodiment, the vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique.

In one embodiment, the method includes supplementing the cultivation with a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The method can provide oxygenation to the growing culture. One embodiment utilizes slow motion of air to remove low-oxygen containing air and introduce oxygenated air. In the case of submerged fermentation, the oxygenated air may be ambient air supplemented daily through mechanisms including impellers for mechanical agitation of liquid, and air spargers for supplying bubbles of gas to liquid for dissolution of oxygen into the liquid.

The method can further comprise supplementing the cultivation with a carbon source. The carbon source is typically a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, canola oil, rice bran oil, olive oil, corn oil, sesame oil, and/or linseed oil; etc. These carbon sources may be used independently or in a combination of two or more.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, sodium chloride, calcium carbonate, and/or sodium carbonate. These inorganic salts may be used independently or in a combination of two or more.

In one embodiment, one or more biostimulants may also be included, meaning substances that enhance the rate of growth of a microorganism. Biostimulants may be species-specific or may enhance the rate of growth of a variety of species.

In some embodiments, the method for cultivation may further comprise adding an antimicrobial in the medium before, and/or during the cultivation process.

In certain embodiments, an antibiotic can be added to a culture at low concentrations to produce microbes that are resistant to the antibiotic. The microbes that survive exposure to the antibiotic are selected and iteratively re-cultivated in the presence of progressively higher concentrations of the antibiotic to obtain a culture that is resistant to the antibiotic. This can be performed in a laboratory setting or industrial scale using methods known in the microbiological arts. In certain embodiments, the amount of antibiotic in the culture begins at, for example, 0.0001 ppm and increases by about 0.001 to 0.1 ppm each iteration until the concentration in the culture is equal to, or about equal to, the dosage that would typically be applied to a livestock animal.

In certain embodiments, the antibiotics are those often used in livestock feed to promote growth and to help treat and prevent illness and infection in animals, such as, for example, procaine, penicillin, tetracyclines (e.g., chlortetracycline, oxytetracycline), tylosin, bacitracin, neomycin sulfate, streptomycin, erythromycin, monensin, roxarsone, salinomycin, tylosin, lincomycin, carbadox, laidlomycin, lasalocid, oleandomycin, virginamycin, and bambermycins. By producing beneficial microbes that are resistant to a particular livestock antibiotic, the microbes can be selected based on which antibiotic may be administered to the animal to treat or prevent a condition. Alternatively, an antibiotic can be selected for a livestock animal based on which beneficial microbe is being administered to the animal according to the subject methods so as not to harm the beneficial microbe.

The pH of the mixture should be suitable for the microorganism of interest. Buffers, and pH regulators, such as carbonates and phosphates, may be used to stabilize pH near a preferred value. When metal ions are present in high concentrations, use of a chelating agent in the medium may be necessary.

The microbes can be grown in planktonic form or as biofilm. In the case of biofilm, the vessel may have within it a substrate upon which the microbes can be grown in a biofilm state. The system may also have, for example, the capacity to apply stimuli (such as shear stress) that encourages and/or improves the biofilm growth characteristics.

In one embodiment, the method for cultivation of microorganisms is carried out at about 5° to about 100° C., preferably, 15 to 60° C., more preferably, 25 to 50° C. In a further embodiment, the cultivation may be carried out continuously at a constant temperature. In another embodiment, the cultivation may be subject to changing temperatures.

In one embodiment, the equipment used in the method and cultivation process is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control undesirable bacterial growth.

In one embodiment, the subject invention further provides a method for producing microbial metabolites such as, for example, biosurfactants, enzymes, proteins, ethanol, lactic acid, beta-glucan, peptides, metabolic intermediates, polyunsaturated fatty acid, and lipids, by cultivating a microbe strain of the subject invention under conditions appropriate for growth and metabolite production; and, optionally, purifying the metabolite. The metabolite content produced by the method can be, for example, at least 20%, 30%, 40%, 50%, 60%, 70%, 80%, or 90%.

The biomass content of the fermentation medium may be, for example, from 5 g/l to 180 g/l or more, or from 10 g/l to 150 g/l. The cell concentration may be, for example, at least 1×10⁹, 1×10¹⁰, 1×10¹¹, 1×10¹²or 1×10¹³ cells per gram of final product.

The microbial growth by-product produced by microorganisms of interest may be retained in the microorganisms or secreted into the growth medium. The medium may contain compounds that stabilize the activity of microbial growth by-product.

The method and equipment for cultivation of microorganisms and production of the microbial by-products can be performed in a batch, a quasi-continuous process, or a continuous process.

In one embodiment, all of the microbial cultivation composition is removed upon the completion of the cultivation (e.g., upon, for example, achieving a desired cell density, or density of a specified metabolite). In this batch procedure, an entirely new batch is initiated upon harvesting of the first batch.

In another embodiment, only a portion of the fermentation product is removed at any one time. In this embodiment, biomass with viable cells, spores, conidia, hyphae and/or mycelia remains in the vessel as an inoculant for a new cultivation batch. The composition that is removed can be a cell-free medium or contain cells, spores, or other reproductive propagules, and/or a combination of thereof. In this manner, a quasi-continuous system is created.

Advantageously, the method does not require complicated equipment or high energy consumption. The microorganisms of interest can be cultivated at small or large scale on site and utilized, even being still-mixed with their media.

### Preparation of Microbe-Based Products

One microbe-based product of the subject invention is simply the fermentation medium containing a microorganism and/or the microbial metabolites produced by the microorganism and/or any residual nutrients. The product of fermentation may be used directly without extraction or purification. If desired, extraction and purification can be easily achieved using standard extraction and/or purification methods or techniques described in the literature.

The microorganisms in the microbe-based product may be in an active or inactive form. Furthermore, the microorganisms may be removed from the composition, and the residual culture utilized. The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

The microbes and/or medium (e.g., broth or solid substrate) resulting from the microbial growth can be removed from the growth vessel and transferred via, for example, piping for immediate use.

In one embodiment, the microbe-based product is simply the growth by-products of the microorganism. For example, biosurfactants produced by a microorganism can be collected from a submerged fermentation vessel in crude form, comprising, for example about 50% pure biosurfactant in liquid broth.

In other embodiments, the microbe-based product (microbes, medium, or microbes and medium) can be placed in containers of appropriate size, taking into consideration, for example, the intended use, the contemplated method of application, the size of the fermentation vessel, and any mode of transportation from microbe growth facility to the location of use. Thus, the containers into which the microbe-based composition is placed may be, for example, from 1 gallon to 1,000 gallons or more. In other embodiments the containers are 2 gallons, 5 gallons, 25 gallons, or larger.

Upon harvesting, for example, the yeast fermentation product, from the growth vessels, further components can be added as the harvested product is placed into containers and/or piped (or otherwise transported for use). The additives can be, for example, buffers, carriers, other microbe-based compositions produced at the same or different facility, viscosity modifiers, preservatives, nutrients for microbe growth, tracking agents, solvents, biocides, other microbes and other ingredients specific for an intended use.

Other suitable additives, which may be contained in the formulations according to the invention, include substances that are customarily used for such preparations. Examples of such additives include surfactants, emulsifying agents, lubricants, buffering agents, solubility controlling agents, pH adjusting agents, preservatives, stabilizers and ultra-violet light resistant agents.

In one embodiment, the product may further comprise buffering agents including organic and amino acids or their salts. Suitable buffers include citrate, gluconate, tartarate, malate, acetate, lactate, oxalate, aspartate, malonate, glucoheptonate, pyruvate, galactarate, glucarate, tartronate, glutamate, glycine, lysine, glutamine, methionine, cysteine, arginine and a mixture thereof. Phosphoric and phosphorous acids or their salts may also be used. Synthetic buffers are suitable to be used but it is preferable to use natural buffers such as organic and amino acids or their salts listed above.

In a further embodiment, pH adjusting agents include potassium hydroxide, ammonium hydroxide, potassium carbonate or bicarbonate, hydrochloric acid, nitric acid, sulfuric acid or a mixture.

In one embodiment, additional components such as an aqueous preparation of a salt, such as sodium bicarbonate or carbonate, sodium sulfate, sodium phosphate, or sodium biphosphate, can be included in the formulation.

Advantageously, in accordance with the subject invention, the microbe-based product may comprise broth in which the microbes were grown. The product may be, for example, at least, by weight, 1%, 5%, l0%, 25%, 50%, 75%, or 100% broth. The amount of biomass in the product, by weight, may be, for example, anywhere from 0% to 100% inclusive of all percentages therebetween.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C. On the other hand, a biosurfactant composition can typically be stored at ambient temperatures.

### Local Production of Microbe-Based Products

In certain embodiments of the subject invention, a microbe growth facility produces fresh, high-density microorganisms and/or microbial growth by-products of interest on a desired scale. The microbe growth facility may be located at or near the site of application. The facility produces high-density microbe-based compositions in batch, quasi-continuous, or continuous cultivation.

The microbe growth facilities of the subject invention can be located at the location where the microbe-based product will be used (e.g., a free-range cattle pasture). For example, the microbe growth facility may be less than 300, 250, 200, 150, 100, 75, 50, 25, 15, 10, 5, 3, or 1 mile from the location of use.

Because the microbe-based product can be generated locally, without resort to the microorganism stabilization, preservation, storage and transportation processes of conventional microbial production, a much higher density of microorganisms can be generated, thereby requiring a smaller volume of the microbe-based product for use in the on-site application or which allows much higher density microbial applications where necessary to achieve the desired efficacy. This allows for a scaled-down bioreactor (e.g., smaller fermentation vessel, smaller supplies of starter material, nutrients and pH control agents), which makes the system efficient and can eliminate the need to stabilize cells or separate them from their culture medium. Local generation of the microbe-based product also facilitates the inclusion of the growth medium in the product. The medium can contain agents produced during the fermentation that are particularly well-suited for local use.

Locally-produced high density, robust cultures of microbes are more effective in the field than those that have remained in the supply chain for some time. The microbe-based products of the subject invention are particularly advantageous compared to traditional products wherein cells have been separated from metabolites and nutrients present in the fermentation growth media. Reduced transportation times allow for the production and delivery of fresh batches of microbes and/or their metabolites at the time and volume as required by local demand.

The microbe growth facilities of the subject invention produce fresh, microbe-based compositions, comprising the microbes themselves, microbial metabolites, and/or other components of the medium in which the microbes are grown. If desired, the compositions can have a high density of vegetative cells or propagules, or a mixture of vegetative cells and propagules.

In one embodiment, the microbe growth facility is located on, or near, a site where the microbe-based products will be used (e.g., a livestock production facility), preferably within 300 miles, more preferably within 200 miles, even more preferably within 100 miles. Advantageously, this allows for the compositions to be tailored for use at a specified location. The formula and potency of microbe-based compositions can be customized for specific local conditions at the time of application, such as, for example, which animal species is being treated; what season, climate and/or time of year it is when a composition is being applied; and what mode and/or rate of application is being utilized.

Advantageously, distributed microbe growth facilities provide a solution to the current problem of relying on far-flung industrial-sized producers whose product quality suffers due to upstream processing delays, supply chain bottlenecks, improper storage, and other contingencies that inhibit the timely delivery and application of, for example, a viable, high cell-count product and the associated medium and metabolites in which the cells are originally grown.

Furthermore, by producing a composition locally, the formulation and potency can be adjusted in real time to a specific location and the conditions present at the time of application. This provides advantages over compositions that are pre-made in a central location and have, for example, set ratios and formulations that may not be optimal for a given location.

The microbe growth facilities provide manufacturing versatility by their ability to tailor the microbe-based products to improve synergies with destination geographies. Advantageously, in preferred embodiments, the systems of the subject invention harness the power of naturally-occurring local microorganisms and their metabolic by-products to improve GHG management.

The cultivation time for the individual vessels may be, for example, from 1 to 7 days or longer. The cultivation product can be harvested in any of a number of different ways.

Local production and delivery within, for example, 24 hours of fermentation results in pure, high cell density compositions and substantially lower shipping costs. Given the prospects for rapid advancement in the development of more effective and powerful microbial inoculants, consumers will benefit greatly from this ability to rapidly deliver microbe-based products.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1-B. amy Growth By-Products

In an exemplary embodiment, B. amy can produce biosurfactants including, e.g., surfactin, fengycin, iturin, bacillomycin, lichenysin, difficidin, and/or a maltose-based glycolipid. These biosurfactants can reduce the interfacial tension between the liquid and solid phases of manure and promote separation thereof. Additionally, one or more of these biosurfactants can inhibit methanogenesis in manure by interfering with the production and/or maintenance of the exopolysaccharide matrix that forms methanogenic bacterial biofilms.

In an exemplary embodiment, B. amy can produce enzymes that are helpful for digestion and composting of manure solid materials, as well as control of methanogenic bacteria, such as:

lignocellulytic enzymes, e.g., cellulose, xylanase, laccase, and manganese catalase, which can enhance digestion of polysaccharides, such as cellulose, xylan, hemicellulose, and lignin, present in manure solids;

digestive enzymes, e.g., amylases, lipases, and proteases (e.g., collagenase-like protease, peptidase E (N-terminal Asp-specific dipeptidase), peptidase s8 (subtilisin-like serine peptidase), serine peptidase, and endopeptidase La), which can increase decomposition of proteins, fats and carbohydrates in manure;

proteinase K (and/or a homolog thereof), which can specifically lyse pseudomurien, a major structural cell wall component of some archaea, including methanogens; and

diglycolic acid dehydrogenase (DGADH), (and/or a homolog thereof), which can disrupt ether bonds between the glycerol backbone and fatty acids of the phospholipid layer of archaeal cell membranes.

In an exemplary embodiment, B. amy can produce organic acids, such as propionic acid, which can disrupt the structure of archaeal cell membrane and stimulate acetogenic microorganisms, which produce acetic acid from hydrogen and carbon dioxide. This results in reduced hydrogen availability for methanogenic microbes to carry out methanogenesis.

### REFERENCES

CHASTAIN, J. P., 2019, “Chapter 4, Solid-Liquid Separation Alternatives for Manure Handling and Treatment”, USDA Environmental Engineering National Engineering Handbook.

Manitoba Agriculture, Food and Rural Development, 2015, “Properties of Manure.” <https://www.gov.mb.ca/agriculture/environment/nutrient-management/pubs/properties-of-manure.pdf>

What is claimed:

1. A method of processing manure, the method comprising applying a microbe-based product comprising a microbial biosurfactant and/or a beneficial microorganism to the manure, wherein the biosurfactant and/or the microorganism promote separation of solids and liquids in the manure, thereby producing a solids fraction and a liquid fraction; and collecting the solids fraction and liquid fraction separately, wherein the separated solids fraction has a moisture content of less than 40% by mass.

2. The method of claim 1, further comprising mixing the microbe-based product with the manure for about 1 minute to 6 hours after the microbe-based product is applied to the manure and, optionally, allowing the mixture to sit undisturbed for 1 hour to 72 hours.

3. The method of claim 1, wherein collection of the solids fraction is performed via one or more methods selected from: centrifuge, hydrocyclone, stationary inclined screens, in-channel flighted conveyor screen, rotating screen, screw press, belt press, and rotary press.

4. The method of claim 1, wherein the biosurfactant is a sophorolipid.

5. The method of claim 4, wherein the sophorolipid is an acidic sophorolipid or a lactonic sophorolipid.

6. The method of claim 1, wherein the biosurfactant-producing microorganism is Bacillus amyloliquefaciens strain NRRL B-67928.

7. The method of claim 1, wherein the biosurfactant-producing microorganism is Bacillus subtilis strain NRRL B-68031.

8. The method of claim 1, wherein greenhouse gas and/or other polluting emissions from the manure are reduced.

9. The method of claim 8, wherein the greenhouse gas and/or other polluting emissions are methane, carbon dioxide, nitrous oxide, ammonia and/or hydrogen sulfide.

10. The method of claim 1, wherein the solids fraction is utilized for composting, as a fertilizer, as a soil amendment, as animal bedding, or as a combustible fuel.

11. The method of claim 1, wherein the liquid fraction is utilized for irrigation of fields, cleaning of animal housing and/or farm equipment, animal drinking water, and/or as a fertilizer.

12. A manure composition comprising a manure solids fraction and a microorganism selected from strain NRRL B-67928 and NRRL B-68031, wherein the manure composition has a moisture content of less than 40% by mass.

13. The manure composition of claim 12, further comprising a sophorolipid biosurfactant.

14. A method for irrigating a crop or field, the method comprising obtaining a manure liquid fraction, applying a biosurfactant to the liquid fraction to produce an irrigation composition, and applying the irrigation composition to the crop or field, wherein the presence of the biosurfactant in the irrigation composition enhances the movement of the irrigation composition throughout soil and enhances water use efficiency.
